SEQUENCE LISTING (10 sequences)

SEQ ID NO: 1 - protein, 5 aa, Artificial Sequence
  Synthetic Peptide
RGDFK

SEQ ID NO: 2 - protein, 5 aa, Artificial Sequence
  Synthetic Peptide
RGDFE

SEQ ID NO: 3 - protein, 5 aa, Artificial Sequence
  Synthetic Peptide
RGDFX

SEQ ID NO: 4 - protein, 5 aa, Artificial Sequence
  Synthetic Peptide
RGDWK

SEQ ID NO: 5 - protein, 5 aa, Artificial Sequence
  Synthetic Peptide
RGDWE

SEQ ID NO: 6 - protein, 5 aa, Artificial Sequence
  Synthetic Peptide
RGDWX

SEQ ID NO: 7 - protein, 5 aa, Artificial Sequence
  Synthetic Peptide
RGDXK

SEQ ID NO: 8 - protein, 5 aa, Artificial Sequence
  Synthetic Peptide
RGDXX

SEQ ID NO: 9 - protein, 5 aa, Artificial Sequence
  Synthetic Peptide
RGDXE

SEQ ID NO: 10 - protein, 5 aa, Artificial Sequence
  Synthetic Peptide
RGDFK

(19) United States
(12) Patent Application Publication
(10) Pub. No.: US 2009/0053280 A1
(43) Pub. Date: Feb. 26, 2009

(54) COATING STENTS WITH CYCLIC RGD PEPTIDES OR MIMETICS

(76) Inventors: Michael Joner, Munich (DE); Horst Kessler, Garching (DE); Renu Virmani, Chevy Chase, MD (US)

Correspondence Address: WOLF GREENFIELD & SACKS, P.C., 600 ATLANTIC AVENUE, BOSTON, MA, 02210-2206, US

(21) Appl. No.: 12/121,568

(22) Filed: May 15, 2008

Related U.S. Application Data
(60) Provisional application No. 60/930,208, filed on May 15, 2007.

(51) Int. Cl. A61L 27/54; A61F 2/82; A61L 27/28

(52) U.S. Cl. 424/423; 427/2.25; 623/1.46

(57) ABSTRACT

It is an object of the present invention to overcome certain disadvantages of stents, such as drug eluting stents. In particular, it is an object to provide a stent with improved re-endothelialization ability and/or simultaneous reduced rate of restenosis and/or minimal or no inflammatory potential.

### RELATED APPLICATIONS

This application claims the benefit of the filing date of U.S. Provisional application 60/930,208, filed May 15, 2007. The entire contents and teachings of the referenced application are expressly incorporated herein in their entirety.

### FIELD OF THE INVENTION

The present invention relates to coated bare metal stents, to methods for coating or producing such coated bare metal stents, and to their use in medicine. In particular, the present invention relates to bare metal stents coated with integrin selective peptides or peptidomimetics, to methods for coating or producing such coated stents, and to their use as implants.

### BACKGROUND OF INVENTION

Approximately 5 million diagnostic coronary catheterizations are performed world-wide every year. About one third of these patients are required to receive any kind of intervention including implantation of coronary bare metal stents. One of the major limitations of this technology is, however, the unusually high rate of neointimal growth within these implants, which results in re-occlusion of the stent implant. Between 20 and 60% of patients receiving bare metal stents are prone to restenosis and need to undergo repeat revascularization. In a postmortem study by Farb et al., the common underlying cause of bare metal stent restenosis were increased inflammation and neoangiogenesis (Farb A. et al., Circulation, 2002), which are both signs that a device lacks biocompatibility. The introduction of drug eluting stents releasing potent antiproliferative compounds into the surrounding vascular tissue resulted in a striking decrease in the need for repeat revascularization procedures due to marked reductions in neointimal growth (Moses, J. W., NEJM, 2003; Stone, G. W., NEJ, 2004). Despite this early clinical success with current drug eluting stents, there have been concerns about an increased risk of late thrombotic events with these stents. It has been shown in preclinical and clinical studies that there is a significant delay in vascular healing following implantation of current drug eluting stents (Camenzind, E., 2007; Finn, A. V., 2005; Joner, M., 2006; Pfisterer, M., 2006). This is likely caused by unspecific anti-proliferative effects of the drugs used on current drug eluting stents, resulting in diminished endothelial regeneration with impaired functionality of the newly formed tissue. Moreover, the polymers used to control the elution of the drugs have been reported to cause chronic inflammatory reactions, thereby increasing the risk of late thrombotic events (Virmani, R., 2004). Part of the antiproliferative potency of drug eluting stents is needed to overcome the hazard of inflammatory responses related to the synthetic polymers used in these stents. Improvements in stent design and biocompatibility are needed to support vascular healing following implantation of these medical implant devices. There is a great need for improved bare metal stent technology allowing to avoid the above-mentioned problems that are associated with the currently-used stent technology.

### SUMMARY OF INVENTION

It is an object of the present invention to overcome certain disadvantages of stents, such as drug eluting stents. In particular, it is an object to provide a stent with improved re-endothelialization ability and/or simultaneous reduced rate of restenosis and/or minimal or no inflammatory potential.

Although it is known that integrin signalling is involved in the body's response to vascular injury, the processes are very complex. In general it is believed that binding of an integrin selective peptide to an integrin on the surface of vascular smooth muscle cells activates the signalling cascade within the cell, triggering the proliferation of the cell, and, thus, promoting restenosis. As a result, currently, approaches to preventing restenosis of, for instance, implanted bare metal stents are likely to involve inhibition of integrin signalling and, consequently, cell proliferation. Contrary thereto, the present inventors have surprisingly discovered that integrin selective peptides coated onto a bare metal stent can prevent the occurrence of restenosis despite activation of integrin signalling. This is believed, without wishing to be bound to theory, to be due to a directed adhesion of endothelial cells to the stent coated with integrin selective peptides, which promote a rapid endothelialization of the stent into the surrounding vascular tissue, thereby counteracting the formation of restenosis.

Described herein are compositions of matter and methods that address problems of currently-used stent technology. The present invention addresses the above object by providing the subject matter specified by the following items 1 to 21:

1. A coated bare metal stent, wherein the coating comprises a chemical entity having the general formula (I)

P-S-A (I),

where

P represents an integrin selective peptide or peptidomimetic;

S is missing or represents a spacer; and

A represents an anchor.

2. The coated bare metal stent according to item 1, wherein the integrin selective peptide or peptidomimetic preferentially binds to alpha v beta 3 integrin.

3. The coated bare metal stent according to item 1 or 2, wherein the integrin selective peptide or peptidomimetic exhibits limited binding affinity to alpha II beta 3 integrin.

4. The coated bare metal stent according to any one of items 1 to 3, wherein the integrin selective peptide is a linear peptide of the general formula (II)

(Xaa)ₙ-RGD-(Xaa)ₘ (II),

wherein each residue Xaa independently represents any natural or unnatural amino acid; R represents arginine; G represents glycine; D represents aspartic acid; n and m each independently are 0, 1, 2, 3, 4 or 5, and S (or A, if S is absent) are bonded to the integrin selective peptide via a covalent bond.

5. The coated bare metal stent according to any one of items 1 to 3, wherein the integrin selective peptide is a cyclic peptide of the general formula (III)

c(-RGD-(Xaa)ₙ-) (III),

wherein each residue Xaa independently represents any natural or unnatural amino acid; R represents arginine; G represents glycine; D represents aspartic acid; n is 0, 1, 2, 3, 4 or 5; c means cyclo, and S (or A, if S is absent) is bonded to the integrin selective peptide via a covalent bond.

6. The coated bare metal stent according to any one of items 1 to 3, wherein the integrin selective peptide is a cyclic peptide of the general formula (IV)

c(-RGD-xaa-Xaa-) (IV),

wherein xaa represents an amino acid in the D-configuration having an aromatic side chain; Xaa represents any natural or unnatural amino acid; R represents arginine; G represents glycine; D represents aspartic acid; and c means cyclo.

7. The coated bare metal stent according to any one of items 1 to 3, wherein the integrin selective peptide is selected from:

c(-Arg-Gly-Asp-phe-Lys-);
c(-Arg-Gly-Asp-phe-Glu-);
c(-Arg-Gly-Asp-phe-Orn-);
c(-Arg-Gly-Asp-trp-Lys-);
c(-Arg-Gly-Asp-trp-Glu-);
and
c(-Arg-Gly-Asp-trp-Orn-).

8. The coated bare metal stent according to any one of items 1 to 3, wherein the peptidomimetic is selected from:

(a) a peptidomimetic of a linear peptide of the general formula (II)

(Xaa)ₙ-RGD-(Xaa)ₘ (II),

wherein each residue Xaa independently represents any natural or unnatural amino acid; R represents arginine; G represents glycine; D represents aspartic acid; n and m each independently are 0, 1, 2, 3, 4 or 5, and S (or A, if S is absent) are bonded to the integrin selective peptide via a covalent bond;
(b) a peptidomimetic of a cyclic peptide of the general formula (III)

c(-RGD-(Xaa)ₙ-) (III),

wherein each residue Xaa independently represents any natural or unnatural amino acid; R represents arginine; G represents glycine; D represents aspartic acid; n is 0, 1, 2, 3, 4 or 5; c means cyclo, and S (or A, if S is absent) are bonded to the integrin selective peptide via a covalent bond;

(c) a peptidomimetic of a cyclic peptide of the general formula (IV)

c(-RGD-xaa-Xaa-) (IV),

wherein xaa represents an amino acid in the D-configuration having an aromatic side chain; Xaa represents any natural or unnatural amino acid; R represents arginine; G represents glycine; D represents aspartic acid; and c means cyclo; and
(d) a peptidomimetic of any of the following integrin selective peptides:

c(-Arg-Gly-Asp-phe-Lys-);
c(-Arg-Gly-Asp-phe-Glu-);
c(-Arg-Gly-Asp-phe-Orn-);
c(-Arg-Gly-Asp-trp-Lys-);
c(-Arg-Gly-Asp-trp-Glu-);
and
c(-Arg-Gly-Asp-trp-Orn-).

9. The coated bare metal stent according to any one of items 1 to 8, wherein the spacer is any organic molecule of sufficient length to allow the integrin selective peptide or peptidomimetic to bind to the integrin with a binding strength of at least 1 to 10% of the binding of the free integrin selective peptide or peptidomimetic (e.g., at least 1, 2, 3, 4, 5, 6, 7, 8, 9 or 10%).

10. The coated bare metal stent according to item 9, wherein the spacer has from 0 to 50 atoms in its backbone.

11. The coated bare metal stent according to any one of items 1 to 10, wherein the anchor is derived from a molecule that comprises a component by which it is able to bind to the surface of the bare metal stent.

12. The coated bare metal stent according to item 11, wherein the anchor is selected from the group consisting of: —W, —V—W, —V—[V—W₂]₂, and —V—[V—(V—W₂)₂]₂, wherein W represents

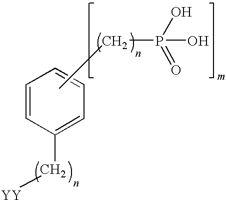

and V represents lysine, aspartic acid or glutamic acid; m is 1, 2 or 3; and n is each independently 1, 2, 3, 4, 5, 6, 7 or 8; YY is an amino or carboxyl group.

13. The coated bare metal stent according to item 11, wherein the anchor is selected from the group consisting of: —CO—CH═CH₂, —CO—(CH)₁₋₂₀—CO—CH═CH₂, —CO—(CH₂)₁₋₂₀—SH, —CO—CH(NH₂)—CH₂—SH, —NH—(CH₂)₁₋₂₀—CO—CH═CH₂, —NH—(CH₂)₂₋₂₀—SH, —NH—CH(CO₂H)—CH₂—SH.

14. The coated bare metal stent according to any one of items 1 to 13 for use as an implant.

15. The coated bare metal stent according to any one of items 1 to 13 for use as an implant to promote blood vessel repair in an individual in need thereof.

16. The coated bare metal stent according to item 14 or 15 in combination with one or more therapeutic agents.

17. A method of coating a bare metal stent or producing a bare metal stent with a coating, comprising the step of coupling the bare metal stent with the chemical entity of the general formula (I).

18. A method of promoting blood vessel repair in an individual in need thereof, comprising introducing into a blood vessel in the individual a coated bare metal stent of any one of items 1 to 13.

19. The method of item 18, wherein the blood vessel is any artery or vein in the body, such as a coronary artery, a peripheral artery, an aorta, an intracerebral vessel, an aneurysm vessel, a renal vessel, a hepatic vessel, or a celiac vessel in the individual.

20. A method of reducing restenosis in an individual in need thereof, comprising introducing into a blood vessel in the individual a coated bare metal stent of any one of items 1 to 13.

21. The method of item 20, wherein the blood vessel is any artery or vein in the body, such as a coronary artery, a peripheral artery, an aorta, an intracerebral vessel, an aneurysm vessel, a renal vessel, a hepatic vessel, or a celiac vessel in the individual.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A and 1B (Example 1): Attachment of HUVECs on nitinol/and or stainless steel coupons following coating with BSA or different concentrations of the cyclic RGD peptide (A) for 1 hour. There was a maximum of attachment observed at 10 μg/mL. To confirm the persistence of cell binding following coating with the RGD peptide, a time course of cell attachment was performed at a concentration of 10 μg/mL (B).

FIG. 2 (Example 2): FAK³⁹⁷ activation following replating of HUVECs onto cyclic RGD, Poly-L-Lysin (PLL) or BSA (CTRL) coated Petri-dishes. Cells were detached, solubilized and replated following coating of the Petri-dishes. FAK activation is only observed in cyclic RGD coated Petri-dishes, which confirms the integrin-specific binding of endothelial cells.

FIGS. 3A-3D (Example 3): Attachment of HUVECs on cyclic RGD coated or BSA coated bare metal stents under static (A, B) and dynamic (C, D) conditions.

FIGS. 4A and 4B: Structure of one of the “cyclic RGD” peptides having a tetraphoshonate anchor used for coating of titanium alloy and/or 316L stainless steel (A); Structure of one of the “cyclic RGD” peptides having a thiol anchor used for coating cobald-chromium (B).

FIGS. 5A-5D (Example 4): Confocal microscopy (A and B) and scanning electron microscopy (C and D) of cyclic RGD-coated and uncoated Nitinol stents 7 days following implantation into rabbit aorta. Endothelial cells were specifically labeled by staining for CD31 (PECAM), longitudinally cut and analyzed by en face microscopy. Green channel=CD31; blue channel=TOTO-3. Electron microscopy in low (15×) and high (200×) magnifications.

FIGS. 6A and 6B (Example 4): Cyclic RGD-coated stents showed greater percentage of endothelialization above and between stent struts both by Scanning Electron Microscopy (A) and Confocal Microscopy assessment of CD31 positive staining (B) 7 days following implantation.

FIG. 7 (Example 5): Bar graph showing the results of migration assays utilizing specialized Millicell culture plates for in vitro testing. HUVECs were seeded onto the inside surface of Millicell membranes and semi-quantified following migration through a microporous membrane at the outside surface after two hours of incubation. There was a dose-dependent increase in migrating endothelial cells when the outside surface of the membrane was coated with cyclic RGD peptide. In the presence of paclitaxel or sirolimus, cellular migration was almost completely abolished with uncoated membranes. However, when the outside membrane was coated with cyclic RGD peptide, there was a significant increase in haplotactic cell migration towards the RGD coated surface.

FIGS. 8A-8F (Example 6): Representative pictures of HUVECs attached to BSA or RGD coated bare metal stents. Endothelial cells were stained for filamental actin (green channel) and focal adhesion kinase (FAK, red channel). TOTO-3 was used as nuclear counter stain (blue channel). The overlay of the green and the red channel results in an orange pseudocolor. When bare metal stents are coated with cyclic RGD peptide (B), there is increased cellular anchorage observed in comparison to BSA coated control stents (A), as seen by the intense orange pseudocolor resulting form focal adhesions in B confirming the integrin-dependent anchorage of endothelial cells. In the presence of paclitaxel (C, D), RGD-coated stents (D) show greater expression of focal adhesions (orange pseudocolor) as compared to BSA coated stents (C), while in the presence of sirolimus, (E, F) cellular attachment is increased via increased in actin fiber formation when bare metal stents are coated with RGD peptide (F) as compared to BSA coated stents (E).

### DETAILED DESCRIPTION

### 1. Definitions

Unless specified otherwise herein, the term “bare metal stent” means a stent or stent graft made of metal, with or without surface modification or surface activation, which contains no polymeric material and is not coated with any polymeric material.

Unless specified otherwise, the term “peptidomimetic” as used herein refers to compounds containing non-peptidic structural elements or being of non-peptide structure, which are capable of mimicking the biological action(s) of a parent peptide. In the context of the present invention, the parent peptide is a peptide, which preferentially binds to an integrin.

Unless specified otherwise herein, the terms “selectively binds to” or “preferentially binds to” mean that the integrin selective peptide or peptidomimetic binds to the indicated molecule(s) or class of molecules with a higher affinity (e.g., at least 10 fold, in certain aspects of the invention: 100 fold) compared to a reference molecule. The reference molecule for an integrin selective peptide or peptidomimetic is any molecule which may interact with a cell, typically by binding to and activating different integrin receptor subtypes. The reference molecule for an alpha v beta 3 integrin selective peptide or peptidomimetic may be any other member of the integrin family, typically alpha II beta 3.

Unless specified otherwise herein, the term “amino acid” encompasses any organic compound comprising at least one amino group and at least one acidic group. The amino acid can be a naturally occurring compound or be of synthetic origin. Preferably, the amino acid contains at least one primary amino group and/or at least one carboxylic acid group. In the context of the present application, the term “amino acid” also refers to residues contained in larger molecules such as peptides and proteins, which are derived from such amino acids and which are bonded to the adjacent residues by means of peptide bonds or peptidomimetic bonds.

Unless specified otherwise herein, the terms “naturally occurring amino acid” and “natural amino acid” encompass amino acid residues encoded by the standard genetic code having the L-configuration and non-standard amino acids, e.g., amino acids having the D-configuration instead of the L-configuration, as well as those amino acids that can be formed by modification of such amino acids, for instance, but not limited thereto, pyroglutamic acid (Glp), norleucine (Nle) and ornithine (Orn).

Unless specified otherwise herein, the terms “unnaturally occurring amino acid” and “unnatural amino acid” encompass amino acid residues having the L- or D-configuration that have not been found in nature, but can be incorporated into a peptide chain. These include, but are not limited to, 4-aminobutyric acid (Abu), 6-aminohexanoic acid (Aha), p-benzoylphenylalanine (Bpa), 2,4-diaminobutyric acid (Dab), 2,3-Diaminiopropionic acid (Dap), homo-cysteine (homo-Cys), homo-phenylalanine (homo-Phe), 2-(indole-3-yl)acetic acid (IAA), 4-(indol-3-yl)butyric acid (IAB), 3-(indol-3-yl)propionic acid (IPA), 1-naphthylalanine (1-Nal), 2-naphtylalanine (2-Nal), phenylglycine (Phg) and 4-halogen-phenylalanine (4-Hal-Phe).

Unless specified otherwise, the “interaction,” “binding” or “binding strength” of an integrin selective peptide or peptidomimetic is determined using standard techniques well known to those skilled in the art, for instance, those described by Goodman, L. et al., 2002 (preferably), and Stragies, R., 2007.

### 2. Integrins

Integrins are a family of heterodimeric transmembrane proteins consisting of an alpha and a beta subunit (Clyman, R. I., 1992). In mammals, 8 beta and 18 alpha subunits combine to form 24 distinct heterodimeric integrins. Although each integrin has its own binding specificity, many bind to the same ligand or to partially overlapping sets of ligands (Clyman, R. I., 1992). The integrins can signal through the cell membrane in either direction: the binding activity of the extracellular matrix is regulated from inside of cell (inside-out signaling) (Shattil, S. J., 1995) and the binding of the extracellular matrix provides intracellular signal transduction (outside-in signaling). Integrin alpha v beta 3/5 is a common receptor that recognizes several extracellular matrix proteins, including osteopontin, vitronectin, thrombospondin, and denatured collagen, to which it binds via an RGD-motif (Arg-Gly-Asp integrin binding motif), which provides strong association between integrin receptors and their ligands (Corjay, M. H., 1999; Stouffer, G. A., 1988).

There is evidence that integrins are involved in the response to vascular injury. Vascular injury is a stimulus for the expression of numerous integrin receptor subtypes and, among other effects, these integrins function in the adhesion of activated platelets to endothelium, white cell/endothelium interactions, platelet-mediated thrombin generation, fibrin clot retraction by nucleated cells, and most important, smooth muscle and endothelial cell proliferation and migration (Sajid, M., 2002). In the normal artery, vascular expression of integrins is generally limited to the luminal endothelial monolayer and is markedly upregulated in both the medial and intimal layer following arterial injury (Corjay, M. H., 1999). However, the precise interaction and regulation of integrin receptors in these processes remains unknown and is of major pertinence during adaptive vascular remodeling. Endothelial cells express membrane-spanning integrins, the receptors for proteins in the subendothelial ECM. Integrins are bound to ECM at specific cellular sites, termed “focal contacts” or “focal adhesions” (Burridge, K., 1989; Burridge, K., 1988) that are responsible for the adhesive interactions of the endothelial cell monolayer with ECM. Integrins on endothelial cells are primarily expressed at the abluminal side (Mehta, D., 2006) and are very important mediators of endothelial functionality. Integrins participate in controlling endothelial cell shape through these protein interactions and transduction of signals in a bidirectional manner from the ECM to the actin cytoskeleton and back.

### 3. Coating of Stent

In one aspect, the present invention relates to a coating whose use results in improved biocompatibility of bare metal stents implanted in the body (e.g., in any blood vessel, such as coronaries). The coating comprises (or consists essentially of or consists of) a chemical entity having the general formula (I)

P-S-A (I),

where

P represents an integrin selective peptide or an integrin selective peptidomimetic;

S is missing or represents a spacer, such as an organic spacer; and

A represents an anchor.

### 3.1 Integrin Selective Peptide or Peptidomimetic

According to the present invention, the integrin selective peptide is a peptide which preferentially binds to an integrin. The integrin selective peptide as part of the coating stimulates cell adhesion to the coated bare metal stent, and thus, the re-endothelialization. In some embodiments of the invention, the integrin selective peptide is a peptide that preferentially binds to one or more of alpha v beta 1, alpha v beta 3, alpha v beta 5 and alpha v beta 6 integrin. In another embodiment non-peptidic integrin ligands are strong and preferred binders to the above-mentioned alpha v integrins.

In certain other embodiments, the integrin selective peptide exhibits no or limited binding affinity (e.g., binding affinity is approximately 100 to 1000 fold lower) to one or more of alpha II beta 3, alpha v beta 1, alpha v beta 5 and alpha v beta 6 integrin. In particular embodiments, the integrin selective peptide exhibits no or limited binding affinity to alpha II beta 3, which contributes to avoiding platelet binding. In particular embodiments, the integrin selective peptide is a peptide that preferentially binds to alpha v beta 3 integrin. Typically, the binding of the peptide to alpha v beta 3 integrin is non-covalent. The binding of the integrin selective peptide to alpha v beta 3 results in the preferential recruitment of endothelial cells.

In certain embodiments, the integrin selective peptide is a linear peptide of the general formula (II)

(Xaa)ₙ-RGD-(Xaa)ₘ (II),

wherein each residue Xaa independently represents any natural or unnatural amino acid; R represents arginine; G represents glycine; D represents aspartic acid; and n and m each independently are 0, 1, 2, 3, 4 or 5. In other embodiments, the integrin selective peptide is a cyclic peptide of the general formula (III)

c(-RGD-(Xaa)ₙ-) (III),

wherein each residue Xaa independently represents any natural or unnatural amino acid; R represents arginine; G represents glycine; D represents aspartic acid; n is 0, 1, 2, 3, 4 or 5; and c means cyclo.

In certain other embodiments, the integrin selective peptide is a cyclic peptide of the general formula (IV)

c(-RGD-xaa-Xaa-) (IV),

wherein xaa represents an amino acid in the D-configuration having an aromatic side chain; Xaa represents any natural or unnatural amino acid; R represents arginine; G represents glycine; D represents aspartic acid; and c means cyclo. In particular embodiments, xaa is D-phenylalanine or D-tryptophan. In particular embodiments Xaa is lysine, glutamic acid or ornithine.

In particular embodiments, the integrin selective peptide is selected from the group consisting of:

| | |
|---|---|
| c(-Arg-Gly-Asp-phe-Lys-); | (SEQ ID NO. 1-9) |
| c(-Arg-Gly-Asp-phe-Glu-); | |
| c(-Arg-Gly-Asp-phe-Orn-); | |
| c(-Arg-Gly-Asp-trp-Lys-); | |
| c(-Arg-Gly-Asp-trp-Glu-); | |
| c(-Arg-Gly-Asp-trp-Orn-); | |
| c(-Arg-Gly-Asp-nal-Lys-); | |
| c(-Arg-Gly-Asp-nal-Orn-); | |
| and | |
| c(-Arg-Gly-Asp-nal-Glu-). | |
| with nal = D-naphthylalanine | |

In other aspects of the invention, the peptide forms a cycle via two side chains thereof. A typical example is a cyclization via the thiol groups of two cystine residues to form a cysteine disulfide bond.

In addition, P in the general formula (I) may also be an integrin selective peptidomimetic. In certain embodiments, the peptidomimetic is a mimetic of one of the parental integrin selective peptides specified herein. Typically, the peptidomimetics of the present invention are derived from the parental integrin selective peptides of the present invention by replacing one or more peptide bonds by one or more functional groups selected from the group consisting of: —CO—NR2-, —NR2-CO—, —CH2-NR2- or —NR2-CH2-, —CO—CHR2-, —CHR2-CO—, —CR2=CR2- and —CR2=CR2-, wherein R2 is H, C1-4 alkyl, phenyl or benzyl or, in the case of peptoid-amino acids, the amino acid side chain of the respective amino acid. In this case, the adjacent Cα does not carry the side chain. Other substituents R2, on the other hand, are present in addition to the side chain attached to Cα. Moreover, if more than one R2 is present, it should be understood that the individual R2s can be the same or different from each other. In particular embodiments, the peptidomimetic is derived from the parental peptide by the introduction of at least one modification selected from the group consisting of

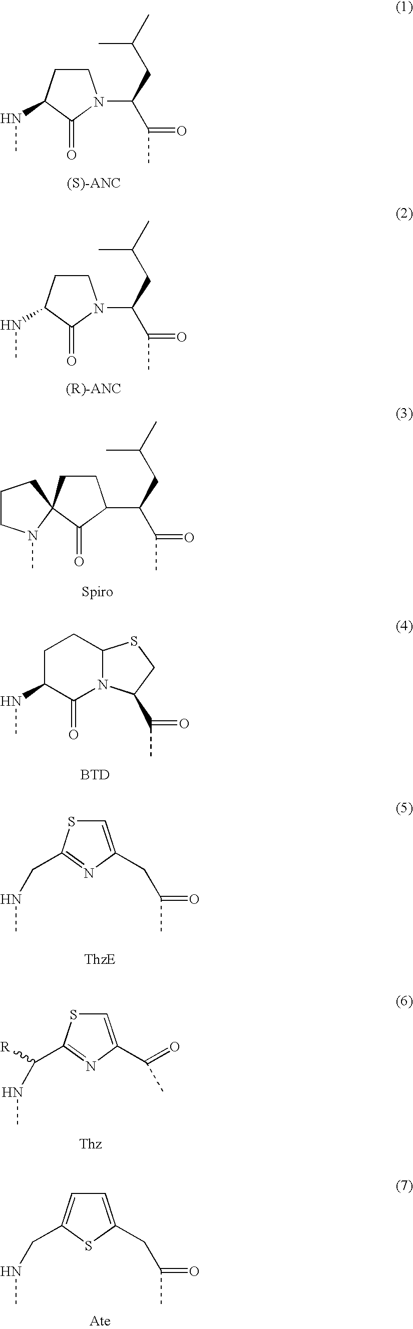

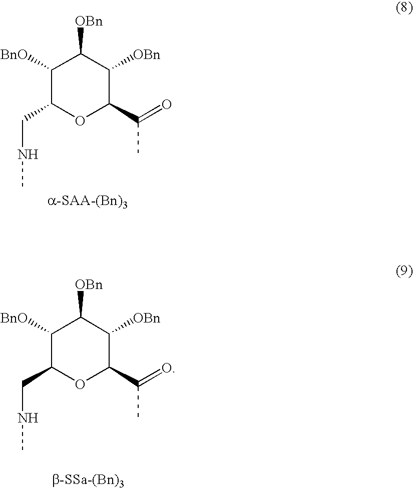

In each moiety above, a dashed line (---) represents a covalent bond. R of Thz (6) can be H, CH₃, phenyl or benzyl.

In specific embodiments, the peptidomimetic is as represented as shown below.

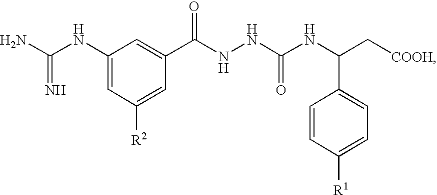

R1 and R2 can be the same or different and are independently selected from H, C1 and CO₂H. In some embodiments, the spacer and/or anchor is attached to the peptidomimetic at R1 or R2, thereby replacing the respective substituent. The spacer/anchor in these aspects of the invention can be selected from CONH(CH₂)n1SH, CO(Ahx)n2Cys, CH₂NH(Ahx)n3CO(CH₂)n4SH, wherein each of n1, n2, n3 and n4 represents an integer independently selected from 1 to 5;

(Ahx=aminohexahoyl; Cys=cysteine)

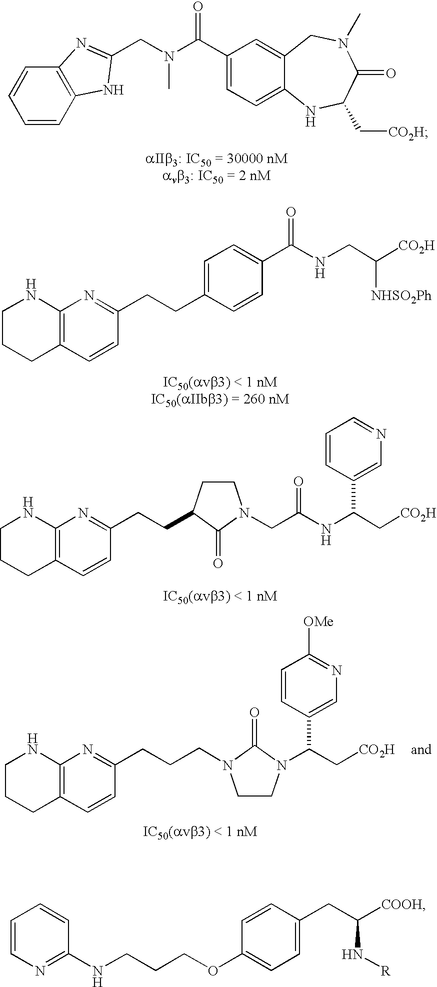

wherein R can be any of the following moieties and is covalently linked at the dashed line represented in each moiety below:

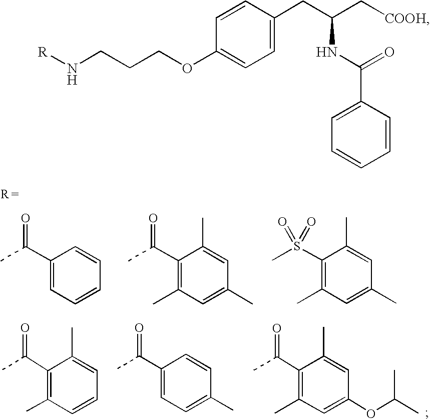

wherein R is a basic group, such as, but not limited to, a basic group comprising one or more nitrogen-containing substituents;

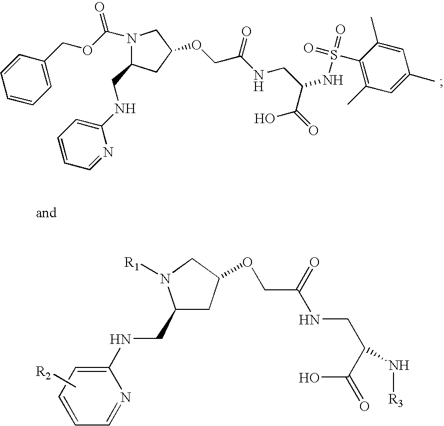

wherein R1, R2, and R3 are independently selected from the following:

R1: —CO₂CH₂Ph, —CO₂Me, —COCH₂C(CH₃)₃, —COCH₂CH₂Ph, or —CH₂CH₂CH₂Ph

### R2: —H, 4-OMe, 4-Cl, or 6-Me; and

R3: —SO₂Ph, —SO₂-2,4,6-trimethylphenyl, —COPh, —CO-2,4,6-trimethylphenyl, or —CO-1-methylcyclohexyl.

Binding of the peptide or peptidomimetic to the spacer (or anchor if the spacer is absent) can be accomplished via reactive functional groups such as thiol groups (e.g., in the side chain of cysteine), carboxyl groups, amino groups, hydroxylamines and triple bonds. The latter two functional groups may undergo ligation via click chemistry with aldehydes (to yield oximes) and azides (to yield triazoles), respectively. The reactive functional groups binding to the spacer (or anchor) may be located at any position of the peptide or peptidomimetic as long as a satisfactory level of preferential binding to the target integrin is maintained. In some aspects of the invention, the reactive functional group is located at one of the Xaa residues of general formulae (II), (III) and (IV).

In the present invention the integrin selective peptides or peptidomimetics can also be present in the form of combinatorial libraries.

### 3.2 Spacer

According to the present invention, the spacer, which is positioned between the integrin selective peptide and the anchor, is any molecule of sufficient length to allow the integrin selective peptide to bind to integrin. In certain embodiments of the invention, the spacer is any organic molecule of sufficient length to allow the integrin selective peptide to bind to integrin. The strength of this binding is typically at least 1 to 10%, and preferably 10%, of the binding of the same peptide (in the free form, without spacer) to the same integrin under the same experimental conditions. The spacer is bonded to the integrin selective peptide or peptidomimetic, for instance, by means of a covalent bond which can be connected to any atom of the integrin selective peptide or peptidomimetic (by substituting a hydrogen atom). In certain embodiments, the spacer is covalently bonded to one of the above-indicated “Xaa”-residues of the integrin selective peptides of general formulae (II) to (IV). Similarly, the spacer is bonded to the anchor, for instance, by means of a covalent bond which is connected to any atom of the anchor (by substituting a hydrogen atom), excluding the atoms of the anchor involved in binding to the metal surface of the stent.

In certain embodiments, the spacer has from 0 to 50 atoms in its backbone. If the surface of the bare metal stent exhibits little or no surface roughness, the use of spacers with at least about 30 backbone atoms may allow to optimize binding of the integrin selective peptide or peptidomimetic to the integrin. If the surface of the bare metal stent is rougher, the number of atoms in the backbone of the spacer can be reduced accordingly.

The spacer should be inert or substantially inert under physiological conditions. It should in particular not be degraded by naturally occurring enzymes.

In particular embodiments, the spacer is selected from the group consisting of

—[CO—(CH₂)x-NH-]p-;

—[CO—CH₂(—O—CH₂CH₂)_{y}—NH-]p—;

—[CO—(CH₂)_{z}—CO—]—, [NH—(CH₂)z-NH—]—;

—[CO—CH₂—(OCH₂CH₂)y-O—CH₂—CO—]—; and

—[NH—CH₂CH₂—(OCH₂CH₂)y-NH—]—.

as well as combinations thereof, wherein p each independently is from 1 to 20; x is from 1 to 12; y each independently is from 1 to 50; and z each independently is from 1 to 12. In certain embodiments, the spacer is one of the above moieties, wherein the value for p is from 1 to 8, the value for x is from 1 to 5, and the values for y and z are each from 1 to 6. It is also possible to employ spacers, wherein one or more of the hydrogen atoms of the above structural formulae is replaced by a substituent. Each substituent can be any chemical entity. In certain embodiments, the substituent can be independently selected from the group consisting of halogen atoms, C1-12 alkyl groups, C1-12 alkoxy groups, C2-12 alkene groups, C2-12 alkyne groups, C3-14 cycloalkyl groups, C3-14 aryl groups, saturated, unsaturated or aromatic 5 to 14-membered heterocyclic groups. The above alkyl groups, alkoxy groups, alkene groups and alkyne groups may be linear, branched or cyclic. They may themselves be substituted, for instance with fluorine atoms.

The spacer itself should not be physiologically active. This is to be considered when choosing possible substituents.

In a specific embodiment, the spacer comprises at least one aminohexanoic acid, such as one, two or three aminohexanoic acids. In another specific embodiment, the spacer comprises at least three consecutive aminohexanoic acids. In another specific embodiment, the spacer is three consecutive aminohexanoic acids.

### 3.3 Anchor

According to the present invention, the anchor is derived from a molecule that comprises a component by which it is able to bind to the surface of the bare metal stent. In those embodiments in which there is an anchor and a spacer, the anchor is joined to the spacer, for instance, by means of a covalent bond which can be connected to any atom of the spacer (by substituting a hydrogen atom).

In certain embodiments, the anchor, before coupling to the surface of the stent, comprises at least one (one or more) phosphoric acid or phosphonic acid. In a specific embodiment, the anchor contains one or more phosphoric acid or phosphonic acid derived moiety. In other specific embodiments, the phosphonic acid derived moiety is an oligomer such as dimer, trimer, tetramer or any other oligomer. In a specific embodiment, the anchor molecule is a tetraphosphonate (Auernheimer, J., 2005). In certain embodiments, the phosphonic acid derived moieties are linked to one another through a series of covalent bonds. In one embodiment, the anchor comprises four phosphonopropionic acids. In another embodiment, the anchor is bis-dibenzylphosphonic acid. In another embodiment, the anchor is based on aromatic phosphonic acids. Suitable anchors of this type may comprise one, two or more 3,5-bisphosphonomethyl-benzoyl (BPMP) moieties, as described in J. Auernheimer and H. Kessler, Bioorg Med Chem Lett. 2006 Jan. 15; 16(2): 271-3. Epub 2005 Oct. 25.

In certain embodiments, the anchor is selected from the group consisting of —W, —V—W, —V—[V—W₂]₂, and —V—[V—(V—W₂)₂]₂, wherein W represents

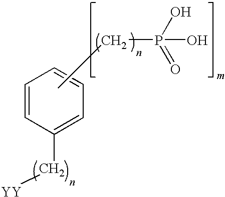

and V represents lysine, aspartic acid or glutamic acid; m is 1, 2 or 3; and n is each independently 1, 2, 3, 4, 5, 6, 7 or 8; YY is an amino or carboxyl group. In particular embodiments, the anchor is one of the group consisting of -Lys-(CO—CH₂—(CH₂)ₙ—PO₃H₂)₂, -Lys-[Lys-(CO—CH₂—(CH₂)ₙ—PO₃H₂)₂]₂ and -Lys-(Lys[-Lys-(CO—CH₂—(CH₂)ₙ—PO₃H₂)₂]₂)₂, wherein n each independently is 0, 1, 2 or 3.

In certain other embodiments, the anchor is selected from the group consisting of —CO—CH═CH₂, —CO—(CH)₁₋₂₀—CO—CH═CH₂, —CO—(CH₂)₁₋₂₀—SH, —CO—CH(NH₂)—CH₂—SH, —NH—(CH₂)₁₋₂₀—CO—CH═CH₂, —NH—(CH₂)₂₋₂₀—SH, —NH—CH(CO₂H)—CH₂—SH. The thiol moiety of the anchor allows for the coupling of the integrin selective peptide to gold plated bare metal stents and Cobalt-chromium derived bare metal stents as well as to bare metal stents made of, but not limited thereto, stainless steel, titanium or titanium alloys.

In other certain embodiments, the anchor is based on acrylic acid functional groups, which allows for coupling to bare metal stents having received a surface modification or activation. Specifically, the acrylic acid functional group may undergo chemical reaction with a free amino group of the modified surface of the stent.

### 3.4 Bare Metal Stent

According to the invention, the coating can be applied to the surface of any bare metal stent. Therefore, in another aspect, the invention relates to a coated bare metal stent, wherein the coating comprises (or, alternatively, consists essentially of or consists of) a compound of the formula (I). In one embodiment, the surface of the bare metal stent may be one selected from the group consisting of ABI alloy (palladium and silver), tantalum, niobium, tungsten, molybdenum, platinum, magnesium, cobalt chromium superalloy, cobalt alloys, titanium alloys, and elgiloy. In another embodiment, the surface of the bare metal stent is stainless steel. In another embodiment, the surface of the bare metal stent is Nitinol. Nitinol, a composition of nickel and titanium, is one of very few alloys that is both superelastic and biocompatible. Thus, nitinol is a material widely used for self-expanding stents.

According to the invention, the bare metal stent may receive a surface modification or activation prior to application of the coating. In certain embodiments, the surface of the stent is treated with an aminofunctionalized silane, providing a suitable anchoring surface for, e.g, acrylic acid functional groups, isothiocyanates (Kalina et al., 2008) (Kalinina, S., Gliemann, H., Lopez-Garcia, M., Auernheimer, J., Schimmel, T., Bruns, M., Schambony, A., Kessler, H., Wedlich, D., “Isothiocyanate-functionalized RGD-peptides as useful alternative in tailoring cell-adhesive surface patterns,” Biomaterials, 2008; 29, p. 3004-3013) or an activated carboxylic group. In other embodiments, the surface is treated with an epoxyfunctionalized silane which allows coupling of a thiol group (under opening the oxirane ring) or other groups for opening the epoxy ring. See e.g., Nanci, A. et al., J Biomed Mat Res, 1998, p. 324-335 or Saargeant, T. D. et al., Biomaterials, 2008; 29, p. 1085-1098.

According to the invention, the bare metal stent can be any form that is available and useful for the location in the body into which it will be introduced, such as any expandable wire form or perforated or non-perforated tube that can be inserted into the body.

### 4. Uses of the Coated Stent

Another aspect of the invention relates to the use of the coated bare metal stent of the invention in medicine, and in particular as an implant. In certain embodiments, the coated bare metal stent is for use as an implant in a blood vessel, the biliary tract, the urinary system, or the lymphatic system. In a particular embodiment, the coated bare metal stent of the invention is for use as an implant to promote blood vessel repair in an individual in need thereof. The blood vessel can be any artery or vein in the body such as a coronary artery, a peripheral artery, an aorta, an intracerebral vessel, an aneurysm vessel, a renal vessel, a hepatic vessel, or a celiac vessel. In a specific embodiment, the stent is for use as an implant in an artery.

The bare metal stent of the invention may also be used together with one or more therapeutic agents. The at least one therapeutic agent may either be provided as part of the stent coating or used (administered) separately to the coated bare metal stent. When administered separately, the therapeutic agent may be administered before or after insertion of the stent or simultaneously. The route of administration may be any appropriate route known in the art. In certain embodiments, the route of administration of the therapeutic agent is systemic, e.g., by the parenteral (including subcutaneous, intramuscular, intravenous or intradermal) route. In particular embodiments, the therapeutic agent is an antiproliferative agent. Examples for antiproliferative agents include, but are not limited to, paclitaxel, sirolimus or derivatives thereof (everolimus, zotarolimus, biolimus, pimecrolimus, tacrolimus). In particular embodiments, the coated bare metal stent is used together with paclitaxel, sirolimus or derivatives thereof. Regarding the other embodiments of the invention, pertaining to bare metal stents that are coated with both the chemical entity of general formula (I) and the at least one therapeutic agent, any way of binding the at least one therapeutic agent to the bare metal stent is conceivable. If the therapeutic agent is an antiproliferative agent, the coupling to the surface of the bare metal stent should be such that the antiproliferative agent is released from the stent surface within a suitable period of time. This can be accomplished by employing chemical groups that are labile under physiological conditions.

In other embodiments, the coated bare metal stent of the invention and the one or more therapeutic agents are arranged in kits, optionally with instructions for use.

### 5. Preparation of the Coated Stent

Another aspect of the invention relates to a method of coating a bare metal stent or producing a bare metal stent with a coating, wherein the coating comprises (or consists of) a chemical entity of the formula (I). According to the invention, the method of coating a bare metal stent or producing a bare metal stent with a coating comprises the step of coupling the bare metal stent with the chemical entity of the general formula (I). The chemical entity of formula (I) may be coupled to the bare metal stent as a whole or coupled via attachment of the anchor, spacer and/or integrin selective peptide or peptidomimetic in any appropriate order and timing (in the sense of a construction kit). In certain embodiments, the anchor and spacer are joined together and coupled first to the bare metal stent followed by attaching the integrin selective peptide or peptidomimetic to the spacer. In certain other embodiments, the anchor is coupled first to the bare metal stent followed by attaching the spacer (if present) to the anchor and then attaching the integrin selective peptide or peptidomimetic to either anchor or spacer (if present).

The step of coupling may comprise the preparation of one or more coating mixtures for application by solubilizing the chemical entity of the formula (I) as a whole or each of anchor, spacer and integrin selective peptide or peptidomimetic, alone or in combination, in an appropriate solvent and contacting the bare metal stent with the thus obtained coating mixture(s).

In certain embodiments, the solubilization is achieved by mixing the chemical entity of the formula (I) as a whole or each of anchor, spacer and integrin selective peptide or peptidomimetic, alone or in combination, with the appropriate solvent by shaking or stirring. Shaking is carried out as needed such as from 3 to 24 hours or overnight. In certain embodiments, the contacting step is carried out under incubation conditions that result in application of the coating to the surface in such a manner that it remains on the surface under the conditions in which the bare metal stent is used. In certain embodiments, the contacting step is carried out at a temperature in the range from +4 to +37° C. In a specific embodiment, the contacting step is carried out at room temperature. In one embodiment, the solvent comprises water. In another embodiment, the solvent comprises PBS. In a specific embodiment, the solvent is a sterile PBS solution at room temperature.

In other embodiments, the method of coating a bare metal stent or producing a bare metal stent with a coating further comprises sterilizing the coated bare metal stent without affecting the binding affinity. In certain embodiments, the sterilization step is carried out before the integrin selective peptide is attached to either anchor or spacer (if present).

In other embodiments, the method of coating a bare metal stent or producing a bare metal stent with a coating further comprises drying the bare metal stent contacted with the appropriate coating mixture(s) for 1 to 24 hours. In a specific embodiment, the bare metal stent contacted with the respective coating mixture(s) is left to dry for 8 to 24 hours. In certain embodiments, the drying step is carried out at a temperature in the range from +4 to +37° C. In a specific embodiment, the drying step is carried out at room temperature.

In certain embodiments, the bare metal stent is covered with the above-mentioned coating mixture at a concentration in the range from 5-100 μg/mL. In a particular embodiment, the concentration is 10 μg/mL. In one embodiment, the concentration of the integrin selective peptide in the coating mixture is at least 10 μg/mL.

The following examples are for purposes of illustration only and are not meant to be limiting in any way.

### EXAMPLE 1

### RGD Peptide Binds to Titanium-Oxide Containing Nitinol Coupons

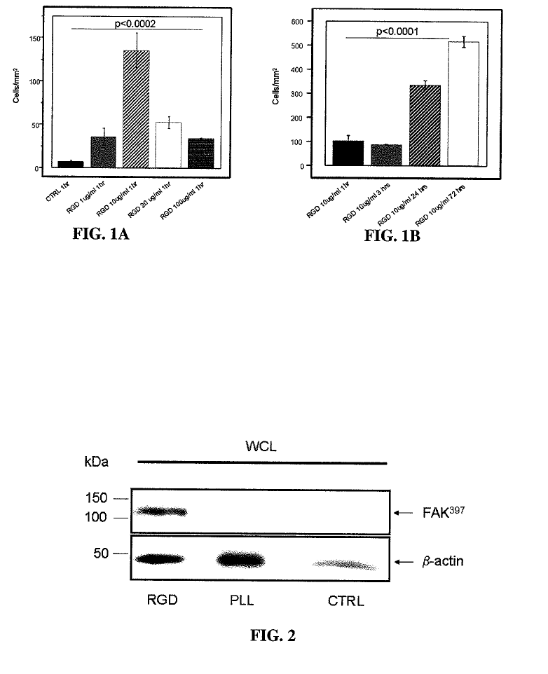

In preliminary experiments, the phosphonate anchored cyclic RGD peptide was shown to specifically bind to Titanium-oxide containing Nitinol coupons. For these experiments, small Nitinol coupons (˜2×2 cm) were coated over night with cyclic RGD peptide solubilized at concentrations of 1, 10, 20 and 100 μg/mL. Applicants found specific binding of RGD peptide over a wide range of doses, with a maximum at 10 μg/mL. Binding of RGD peptide was indirectly proven in a cell adhesion experiment utilizing human umbilical endothelial vein cells (HUVECs). Cells were seeded on RGD-coated and control (BSA coated) coupons at a concentration of approximately 1×10⁵/mL for 1 hour, 3 hours, 24 hours and 72 hours and attachment of the cells was quantified following immunofluorescent labeling and counting the number of cells per area in 6 randomly selected regions. See FIGS. 1A and 1B.

### EXAMPLE 2

### Cyclic RGD Peptide Works through Specific Activation of Integrins

In a separate analysis, applicants focused on the signal pathways that are involved in the process of cellular attachment and anchorage following coating with RGD peptide. It has been reported that the currently used RGD peptide is highly specific for alpha v beta 3 integrin (Meyer, J., 2006) which is abundantly expressed on various cell types. Importantly, endothelial cells express large amounts of alpha v beta 3 integrin, necessary for cellular adhesion, proliferation and migration (Cheresh, D. A., 1987). Upon binding to RGD peptide, integrins form cluster and allow the binding of focal adhesion kinase (FAK), which gets activated via phosphorylation of distinct tyrosine groups. In the current experiment applicants have proven the hypothesis that specific binding of alpha v beta 3 integrin to immobilized RGD peptide results in intracellular activation of focal adhesion kinase (FAK), thus demonstrating an approach to prove the specificity of RGD peptide binding to endothelial cells. See FIG. 2.

### EXAMPLE 3

### Advanced Attachment of Endothelial Cells on RGD Coated Stents In Vitro

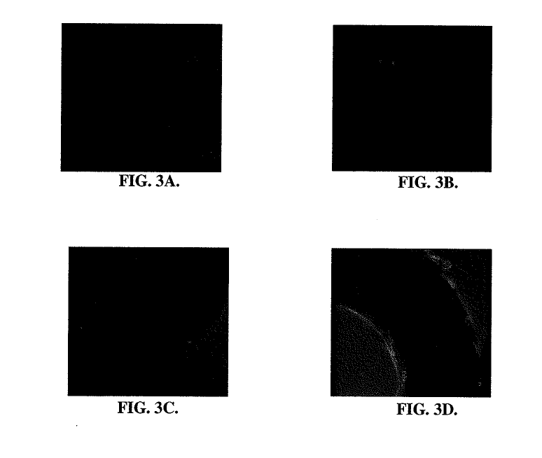

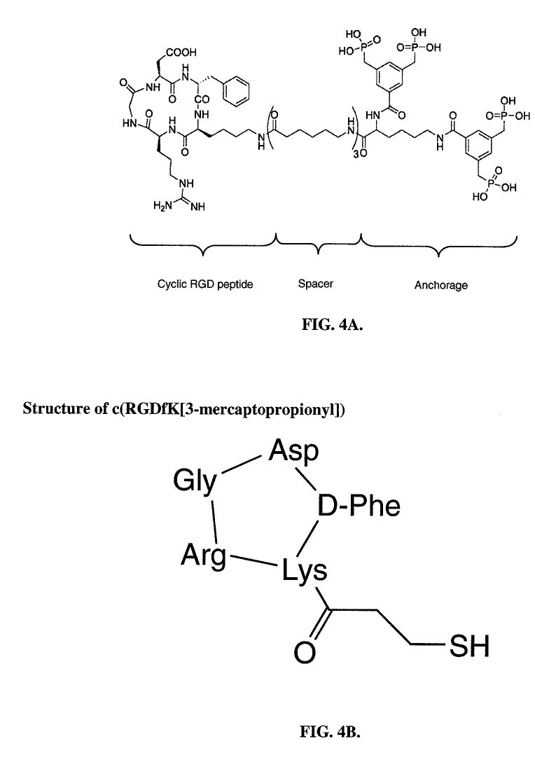

To prove the concept of advanced binding of endothelial cells to RGD coated stents, in vitro experiments were conducted both under static and dynamic conditions. Cells were seeded on Nitinol stents coated with RGD peptide or BSA (control) under static conditions for 3 hours. Following fixation of cells and immunofluorescent staining with Alexa-Fluor phalloidin, the number of adherent endothelial cells were quantified in 3 randomly selected regions. For dynamic adhesion assays, Nitinol stents were cut into small stripes and inserted into customized Ibidi μ-Slides™ (Ibidi, Germany). Flow-through was accomplished for 24 hours and the number of adherent cells quantified. There was significantly greater adherence of endothelial cells to RGD coated stents, as compared to control stents. See FIGS. 3A-3D.

### EXAMPLE 4

### Advanced Attachment of Endothelial Cells on RGD Coated Stents In Vivo

In a study utilizing juvenile New Zealand White Rabbits, applicants have proven the concept that RGD-peptide coating is effective in promoting arterial repair on polymer-free self-expanding stents. The aim of this experimental study was to develop an easy and practical coating for Titanium-containing implants with a specific cyclic RGD peptide by using a new anchor system. It has already been shown that phosphonic acid groups bind strongly over a large pH range (pH 1-9) to TiO₂ and are then distributed on the Titanium surface (Auernheimer, J., 2005). To improve binding to the Titanium surface by the multimer effect, applicants synthesized an anchor block that consisted of four phosphonopropionic acids linked together by a branching unit that was made up of three Lysin residues. The anchor blocks were conjugated with the cyclo (-RGDfK-) peptide (Haubner, R., 1999). They were bridged by a spacer that consisted of three aminohexanoic acids that provided sufficient distance between the peptide and the surface during integrin recognition. This conjugate allows a simple one step coating of the Titanium surface with the peptide.

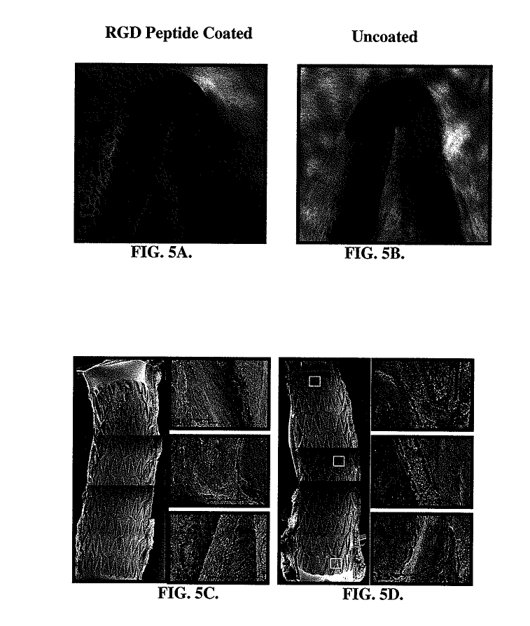

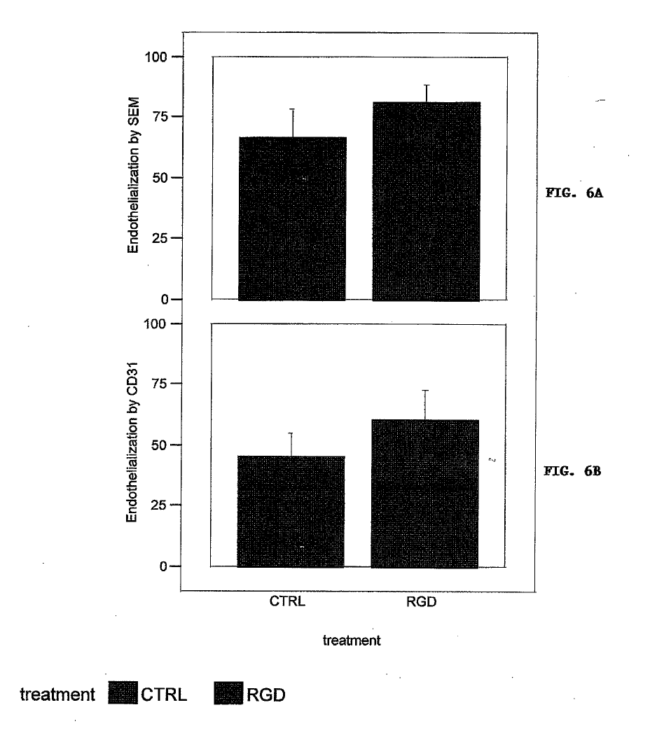

Within this study, RGD-coated Nitinol stents were compared to uncoated Nitinol control stents, which were implanted into the thoracic aorta of New Zealand White Rabbits for 7 days. Re-endothelialization was assessed by confocal and scanning electron microscopy (SEM) and compared among groups. RGD-coated stents showed significantly greater re-endothelialization as compared to uncoated stents (see FIGS. 5A and 5B).

### EXAMPLE 5

### Migration of HUVECs on RGD-Coated Nitinol Coupons in the Presence of Antiproliferative Secondary Drugs

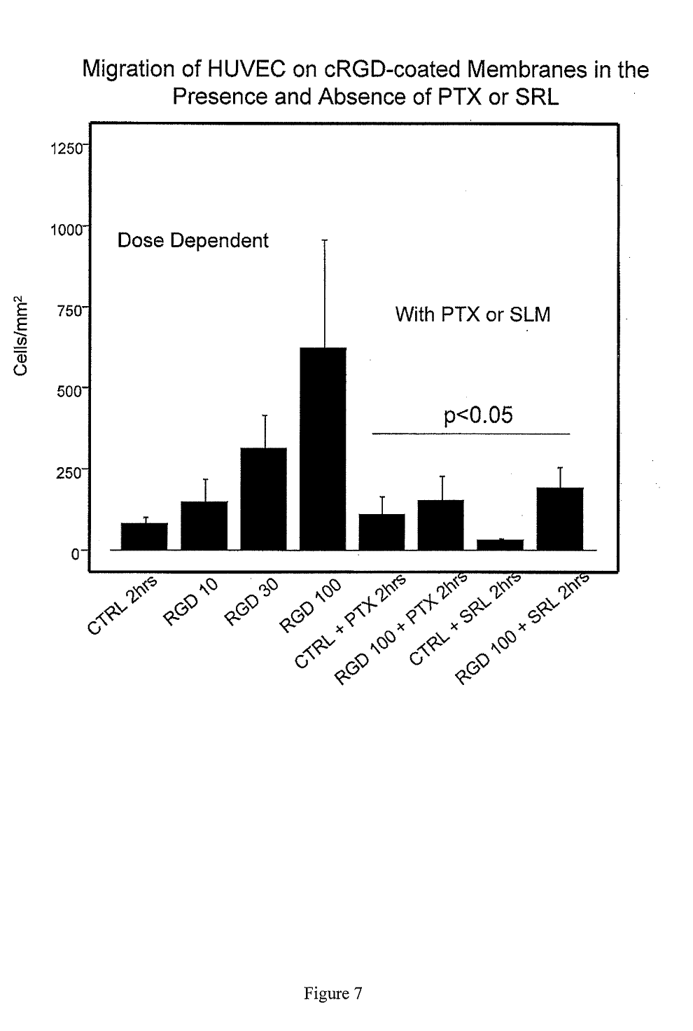

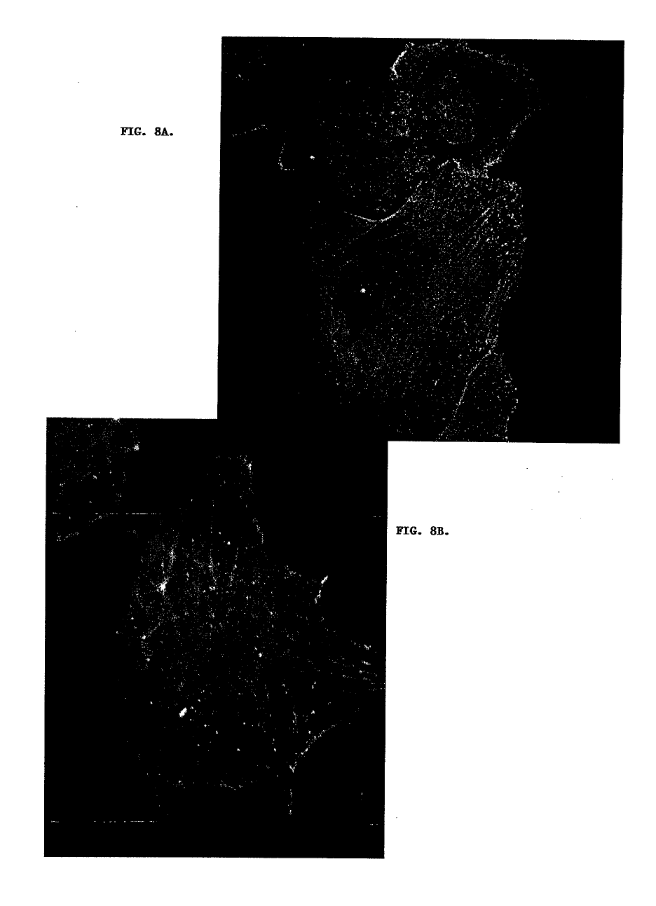

Millicell culture plate inserts from Millipore were coated with cyclic RGD peptide at the outside surface of the microporous membrane only to stimulate haplotactic cell migration. Human umbilical vein endothelial cells (HUVCEs) were loaded into millicell chambers in completed medium in the presence or absence of paclitaxel or sirolimus, and subsequently incubated for 2 hours at 37 C.°. There was a dose-dependent increase in migrating endothelial cells towards the RGD-coated surface of the millicell membrane, with a concentration of 100 μg/ml showing a maximum of cell migration. Importantly, in the presence of antiproliferative drugs (paclitaxel or sirolimus), there was a marked decrease in cell migration. However, the number of migrating endothelial cells were significantly greater when the outside surface of the millicell membrane was coated with cyclic RGD peptide confirming the potent haplotactic stimulus for endothelial cells (FIG. 7).

### EXAMPLE 6

### Advanced Attachment of Endothelial Cells on Bare Metal Stents In Vitro in the Presence of Antiproliferative Compounds

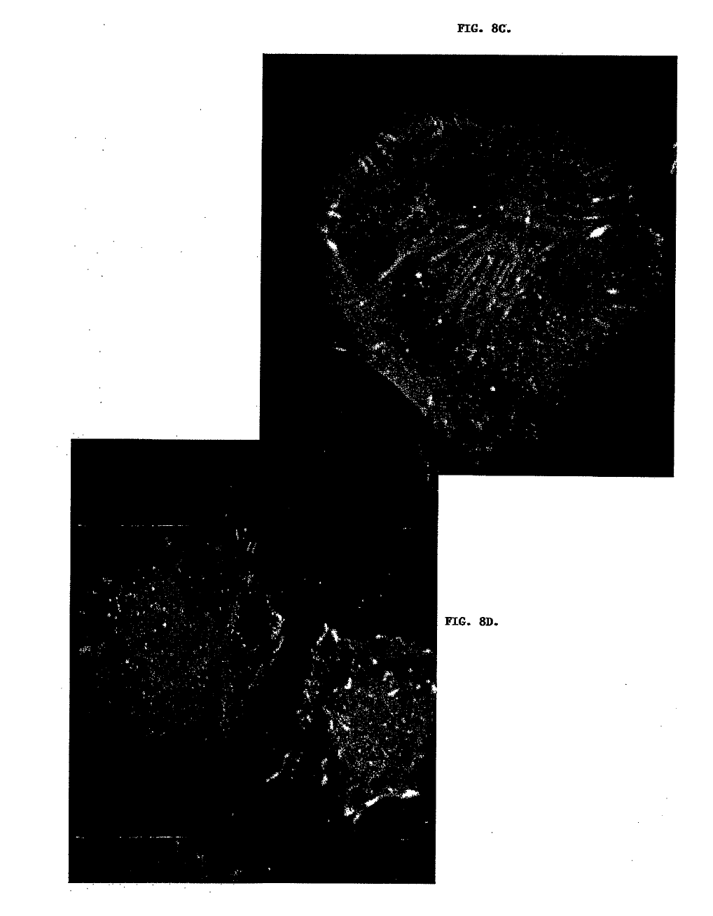

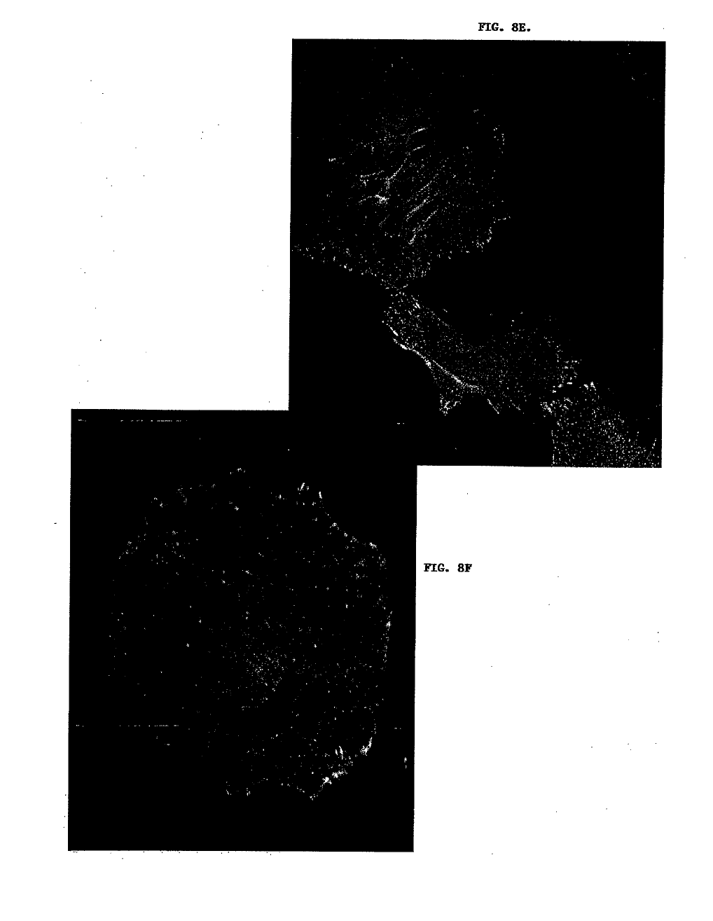

To prove the concept of advanced binding of endothelial cells to RGD coated bare metal stents, further in vitro experiments were conducted. Cells were seeded on Cobald-chromium bare metal stents coated with cyclic RGD peptide or BSA (control) under static conditions for 3 hours. Following fixation of cells, special dual immunofluorescent staining was performed to detect focal adhesions utilizing antibodies against focal adhesion kinase (FAK) and Alexa-Fluor phalloidin to stain for filamental actin (FIGS. 8A to 8F). RGD-coated stents showed greater endothelial cell anchorage along with intense staining for focal adhesions. In the presence of antiproliferative agents (paclitaxel and sirolimus) coating with RGD peptide resulted in greater cellular anchorage and intense staining for focal adhesions compared to BSA coated stents confirming an improved attachment of endothelial cells in the presence of cyclic RGD peptide.

Having thus described several aspects of at least one embodiment of this invention, it is to be understood that the invention pertains to all possible combinations of the above-described individual aspects of the elements of the invention, namely integrin selective peptide or peptidomimetic, spacer, anchor and bare metal stent, falling within the scope of the appended claims.

Furthermore, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### REFERENCES

1. CAMENZIND et al., “Stent thrombosis late after implantation of first-generation drug-eluting stents: a cause for concern,” Circulation, 115(11):1440-55 (2007)
2. FINN et al., “Differential response of delayed healing and persistent inflammation at sites of overlapping sirolimus- or paclitaxel-eluting stents,” Circulation 112(2):270-8 (2005)
3. JONER et al., “Pathology of drug-eluting stents in humans: delayed healing and late thrombotic risk,” J Am Coll Cardiol. 48(1):193-202 (2006)
4. PFISTERER et al., “Late clinical events after clopidogrel discontinuation may limit the benefit of drug-eluting stents: an observational study of drug-eluting versus bare-metal stents,” J Am Coll Cardiol. 48(12):2584-91 (2006)
5. VIRMANI et al., “Localized hypersensitivity and late coronary thrombosis secondary to a sirolimus-eluting stent: should we be cautious?,” Circulation. 109(6):701-5 (2004)
6. BILDER et al., “Restenosis following angioplasty in the swine coronary artery is inhibited by an orally active PDGF-receptor tyrosine kinase inhibitor, RPR101511A,” Circulation 99:(25):3292-9 (1999)
7. RAINES et al., “Smooth muscle cells and the pathogenesis of the lesions of atherosclerosis,” Br. Heart J. 69(1):S30-7 (1993)
8. GIANCOTTI, “A structural view of integrin activation and signaling,” Dev. Cell 4(2):149-51 (2003)
9. GIANCOTTI et al., “Integrin signaling,” Science 285(5430):1028-32 (1999)
10. CLYMAN et al., “Beta 1 and beta 3 integrins have different roles in the adhesion and migration of vascular smooth muscle cells on extracellular matrix,” Exp. Cell. Res. 200(2):272-84 (1992)
11. SHATTIL et al., “Function and regulation of the beta 3 integrins in hemostasis and vascular biology,” Thromb. Haemost. 74(1):149-55 (1995)
12. CHERESH et al., “Human endothelial cells synthesize and express an Arg-Gly-Asp-directed adhesion receptor involved in attachment to fibrinogen and von Willebrand factor,” Proc. Natl. Acad. Sci., USA 84(18):6471-5 (1987)
13. MURPHY et al., “The vitronectin receptor (alpha v beta 3) is implicated, in cooperation with P-selectin and platelet-activating factor, in the adhesion of monocytes to activated endothelial cells,” Biochem. J. 304:537-42 (1994) part 2
14. HUANG et al., “Upregulation of integrins alpha v beta 3 and alpha v beta 5 on human monocytes and T lymphocytes facilitates adenovirus-mediated gene delivery,” J. Virol. 69(4):2257-63 (1995)
15. STERN et al., “Human monocyte-derived macrophage phagocytosis of senescent eosinophils undergoing apoptosis. Mediation by alpha v beta 3/CD36/thrombospondin recognition mechanism and lack of phlogistic response,” Am. J. Pathol., 149(3):911-21 (1996)
16. HALL et al., “Apoptotic neutrophils are phagocytosed by fibroblasts with participation of the fibroblast vitronectin receptor and involvement of a mannose/fucose-specific lectin,” J. Immunol. 153(7):3218-27 (1994).
17. YUE et al., “Osteopontin-stimulated vascular smooth muscle cell migration is mediated by beta 3 integrin,” Exp. Cell Res. 214(2):459-64 (1994)
18. ALBELDA et al., “Integrins and other cell adhesion molecules,” Faseb. J., 4(11): 2868-80 (1990)
19. LIAW et al., “The adhesive and migratory effects of osteopontin are mediated via distinct cell surface integrins. Role of alpha v beta 3 in smooth muscle cell migration to osteopontin in vitro,” J. Clin. Invest. 95(2):713-24 (1995)
20. SRIVATSA et al., “Selective alpha v beta 3 integrin blockade potently limits neointimal hyperplasia and lumen stenosis following deep coronary arterial stent injury: evidence for the functional importance of integrin alpha v beta 3 and osteopontin expression during neointima formation,” Cardiovasc. Res. 36(3):408-28 (1997)
21. AUERNHEIMER et al., “Benzylprotected aromatic phosphonic acids for anchoring peptides on titanium,” Bioorg Med. Chem. Lett. 16:271-273 (2006)
22. AUERNHEIMER et al., “Titanium Implant Materials with Improved Biocompatibility via Coating with Cyclic RGD-Peptides via Phosphonates” Chem Bio Chem. 6:2034-2040 (2005)
23. DUERIG et al., “An Overview of Superelastic Stent Design,” Min Invas Ther & Allied Technol. 9:235-246 (2000)
24. KANTLEHNER et al., “Selective RGD-Mediated Adhesion of Osteoblasts at Surfaces of Implants,” Angew. Chem. Int. Ed. 38:560-562 (1999)
25. KANTLEHNER et al., “Surface Coating with Cyclic RGD Peptides Stimu lates Osteoblast Adhesion and Proliferation as well as Bone Formation,” Chem Bio Chem 1: 107-114 (2000)
26. AUERNHEIMER, et al., “Radio-analytical determination of the coating efficiency of cyclic RGD-peptides,” Helv. Chim. Acta 89:833-840 (2006)
27. MEYER et al., “Targeting RGD Recognizing Integrins: Drug Development, Biomaterial Research, Tumor Imaging and Targeting,” Curr. Pharmaceutical Design 12(22):2723-2747 (2006)
28. PECHY, Chem. Soc. Chem. Commun. 65-66 (1995)
29. HAUBNER et al., “Radiolabeled alpha(v)beta3 integrin antagonists: a new class of tracers for tumor targeting,” J Nucl. Med, 40(6):1061-71 (1999)
30. FARB, et al., “Morphological Predictors of Restenosis After Coronary Stenting in Humans” Circulation (2002) 105:2974-2980.
31. FARB, et al., “Oral Everolimus Inhibits In-Stent Neointimal Growth” Circulation (2002) 106: 2379-2384.
32. MOSES, et al., “Sirolimus-Eluting Stents versus Standard Stents in Patients with Stenosis in a Native Coronary Artery” N Engl J Med (2003) 349(14): 1315-23.
33. STONE, et al., “A Polymer-Based, Paclitaxel-Eluting Stent in Patients with Coronary Artery Disease” N Engl J Med (2004) 350(3): 221-31.
34. AOKI et al., Mol Biol Cell (2005) 16(5):2207-17.
35. SAJID et al., Arterioscler Thromb Vasc Biol (2002) 22(12): 1984-9.
36. MEHTA and MALIK “Signaling Mechanisms Regulating Endothelial Permeability” Physiol Rev (2006) 86(1):279-367.
37. NANCI et al., J Biomed Materials Research Part A (1998) 40(2):324-325.
38. SARGEANT et al., Biomaterials (2008) 29(8):1085-98.
39. KALININA et al., Biomaterials (2008) 29(20):3004-13.

1. A coated bare metal stent, wherein the coating comprises a chemical entity having the general formula (I)
P-S-A (I)
where
P represents an integrin selective peptide or peptidomimetic;
S is missing or represents a spacer; and
A represents an anchor.

2. The coated bare metal stent according to claim 1, wherein the integrin selective peptide preferentially binds to alpha v beta 3 integrin.

3. The coated bare metal stent according to claim 1, wherein the integrin selective peptide exhibits limited binding affinity to alpha II beta 3 integrin.

4. The coated bare metal stent according to claim 1, wherein the integrin selective peptide is a linear peptide of the general formula (II)
(Xaa)ₙ-RGD-(Xaa)ₘ (II)
wherein each residue Xaa independently represents any natural or unnatural amino acid; R represents arginine; G represents glycine; D represents aspartic acid; n and m each independently are 0, 1, 2, 3, 4 or 5, and S (or A, if S is absent) is bonded to the integrin selective peptide via a covalent bond.

5. The coated bare metal stent according to claim 1, wherein the integrin selective peptide is a cyclic peptide of the general formula (III)
c(-RGD-(Xaa)ₙ-) (III)
wherein each residue Xaa independently represents any natural or unnatural amino acid; R represents arginine; G represents glycine; D represents aspartic acid; n is 0, 1, 2, 3, 4 or 5; c means cyclo, and S (or A, if S is absent) is bonded to the integrin selective peptide via a covalent bond.

6. The coated bare metal stent according to claim 1, wherein the integrin selective peptide is a cyclic peptide of the general formula (IV)
c(-RGD-xaa-Xaa-) (IV),
wherein xaa represents an amino acid in the D-configuration having an aromatic side chain;
Xaa represents any natural or unnatural amino acid; R represents arginine; G represents glycine; D represents aspartic acid; and c means cyclo, and S (or A, if S is absent) is bonded to the integrin selective peptide via a covalent bond.

7. The coated bare metal stent according to claim 1, wherein the integrin selective peptide is selected from:

| | |
|---|---|
| c(-Arg-Gly-Asp-phe-Lys-); | (SEQ ID NO: 1) |
| c(-Arg-Gly-Asp-phe-Glu-); | (SEQ ID NO: 2) |
| c(-Arg-Gly-Asp-phe-Orn-); | (SEQ ID NO: 3) |
| c(-Arg-Gly-Asp-trp-Lys-); | (SEQ ID NO: 4) |
| c(-Arg-Gly-Asp-trp-Glu-); | (SEQ ID NO: 5) |
| and | |
| c(-Arg-Gly-Asp-trp-Orn-). | (SEQ ID NO: 6) |

8. The coated bare metal stent according to claim 1, wherein the peptidomimetic is selected from:
(a) a peptidomimetic of a linear peptide of the general formula (II)
(Xaa)ₙ-RGD-(Xaa)ₘ (II),
wherein each residue Xaa independently represents any natural or unnatural amino acid; R represents arginine; G represents glycine; D represents aspartic acid; n and m each independently are 0, 1, 2, 3, 4 or 5, and S (or A, if S is absent) are bonded to the integrin selective peptide via a covalent bond;
(b) a peptidomimetic of a cyclic peptide of the general formula (III)
c(-RGD-(Xaa)ₙ-) (III),
wherein each residue Xaa independently represents any natural or unnatural amino acid; R represents arginine; G represents glycine; D represents aspartic acid; n is 0, 1, 2, 3, 4 or 5; c means cyclo, and S (or A, if S is absent) are bonded to the integrin selective peptide via a covalent bond;
(c) a peptidomimetic of a cyclic peptide of the general formula (IV)
c(-RGD-xaa-Xaa-) (IV),
wherein xaa represents an amino acid in the D-configuration having an aromatic side chain; Xaa represents any natural or unnatural amino acid; R represents arginine; G represents glycine; D represents aspartic acid; and c means cyclo; and
(d) a peptidomimetic of any of the following integrin selective peptides:

| | |
|---|---|
| c(-Arg-Gly-Asp-phe-Lys-); | (SEQ ID NO: 1) |
| c(-Arg-Gly-Asp-phe-Glu-); | (SEQ ID NO: 2) |
| c(-Arg-Gly-Asp-phe-Orn-); | (SEQ ID NO: 3) |
| c(-Arg-Gly-Asp-trp-Lys-); | (SEQ ID NO: 4) |
| c(-Arg-Gly-Asp-trp-Glu-); | (SEQ ID NO: 5) |
| and | |
| c(-Arg-Gly-Asp-trp-Orn-). | (SEQ ID NO: 6) |

9. The coated bare metal stent according to claim 1, wherein the spacer is any organic molecule of sufficient length to allow the integrin selective peptide or peptidomimetic to bind to the integrin with a binding strength of at least 1 to 10% of the binding of the free integrin selective peptide or peptidomimetic.

10. The coated bare metal stent according to claim 9, wherein the spacer has from 0 to 50 atoms in its backbone.

11. The coated bare metal stent according to claim 1, wherein the anchor is derived from a molecule that comprises a component by which it is able to bind to the surface of the bare metal stent.

12. The coated bare metal stent according to claim 11, wherein the anchor is selected from the group consisting of: —W, —V—W, —V—[V—W₂]₂, and —V—[V—(V—W₂)₂]₂, wherein W represents 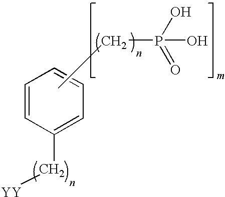
and V represents lysine, aspartic acid or glutamic acid; m is 1, 2 or 3; and n each independently is 1, 2, 3, 4, 5, 6, 7 or 8; YY is an amino or carboxyl group.

13. The coated bare metal stent according to claim 11, wherein the anchor is selected from the group consisting of —CO—CH═CH₂, —CO—(CH)₁₋₂₀—CO—CH═CH₂, —CO—(CH₂)₁₋₂₀—SH, —CO—CH(NH₂)—CH₂—SH, —NH—(CH₂)₁₋₂₀—CO—CH═CH₂, —NH—(CH₂)₂₋₂₀—SH, —NH—CH(CO₂H)—CH₂—SH.

14. The coated bare metal stent according to claim 1 for use as an implant.

15. The coated bare metal stent according to claim 1 for use as an implant to promote blood vessel repair in an individual in need thereof.

16. The coated bare metal stent according to claim 14 in combination with one or more therapeutic agents

17. A method of coating a bare metal stent or producing a bare metal stent with a coating, comprising the step of coupling the bare metal stent with the chemical entity of the general formula (I).

18. A method of promoting blood vessel repair in an individual in need thereof, comprising introducing into a blood vessel in the individual a coated bare metal stent of claim 1.

19. The method of claim 18, wherein the blood vessel is any artery or vein in the body, such as a coronary artery, a peripheral artery, an aorta, an intracerebral vessel, an aneurysm vessel, a renal vessel, a hepatic vessel, or a celiac vessel in the individual.

20. A method of reducing restenosis in an individual in need thereof, comprising introducing into a blood vessel in the individual a coated bare metal stent of claim 1.

21. The method of claim 20, wherein the blood vessel is any artery or vein in the body, such as a coronary artery, a peripheral artery, an aorta, an intracerebral vessel, an aneurysm vessel, a renal vessel, a hepatic vessel, or a celiac vessel in the individual.
